(19) United States
(12) Patent Application Publication
(10) Pub. No.: US 2020/0251217 A1
(43) Pub. Date: Aug. 6, 2020

(54) Diagnosis Method Using Image Based Machine Learning Analysis of Handwriting

(71) Applicants: Renee CASSUTO, Miami, FL (US); James CASSUTO, Miami, FL (US)

(72) Inventors: Renee CASSUTO, Miami, FL (US); James CASSUTO, Miami, FL (US)

(21) Appl. No.: 16/854,297

(22) Filed: Apr. 21, 2020

Related U.S. Application Data
(60) Provisional application No. 62/947,186, filed on Dec. 12, 2019.

(51) Int. Cl. G16H 50/20; G06N 20/00; G16H 50/30; G06K 9/00

(52) U.S. Cl. CPC G16H 50/20; G06K 9/00422; G16H 50/30; G06N 20/00

(57) ABSTRACT

Handwriting analysis is provided by data analysis using machine learning. A handwriting sample is received and the sample is analyzed by one or more analysis components that can include one or more of: segmentation analysis of handwriting with numeric extraction of data, vector analysis of handwriting, demographic data, known diagnoses, data from other manual/motor tasks, and data from other cognitive/higher function tasks. Machine learning is used to adjust or add criteria in at least one of the analysis components, the machine learning comprising a predicted probability of diagnosis based on prior handwriting analysis samples.

### RELATED APPLICATION

The present Patent Application claims priority to Provisional Patent Application No. 62/947,186, filed Dec. 12, 2019, filed by the inventors hereof and which is incorporated by reference herein.

### BACKGROUND

### Technical Field

The present invention relates generally to the examination, analysis, and diagnosis of health and illness conditions based on handwriting samples, and skills related to handwriting. More particularly the invention relates to the use of machine learning and artificial intelligence technology in the analysis, diagnosis, and treatment of said conditions of health and illness, and in the analysis of handwriting skills.

### Background Art

The term “handwriting” as referred herein, should be interpreted in a large sense to encompass any writing or drawing activities using any medium. This may include, but is not limited to, pen to paper or electronic acquisition (i.e., touch screen tablet) for writing letters, writing numbers, drawing simple figures, drawing complex figures, and/or any combination of the aforementioned.

Handwriting analysis, sometimes called “graphology”, is a known art, but has traditionally been used as a form of psychoanalysis, as well as occasionally for a variety of pseudoscience and entertainment purposes. A significant aspect of handwriting analysis is in the matching of a handwriting sample to the handwriting of an individual, either from known samples or from familiarity. Within the education sector handwriting analysis is also used to find students with sub-par handwriting who may then benefit from intervention (more assignments, therapy session, remediation).

Of note, handwriting is a neuromuscular product, so apart from other aspects, handwriting can be used in the analysis of neuromuscular conditions. An issue with use of such analysis, as well as with other aspects of handwriting analysis, is that handwriting is a complex affect, and it is not always possible for even a trained or experienced individual to recognize particular characteristics. Along the same lines, any single characteristic could be the result of the author's or subject's general writing style. Conversely, a particular gesture could be indicative of a neuromuscular condition or could be part of the subject's general style or could be a separate effect.

Handwriting proficiency requires fine motor control, bilateral and visual motor integration, motor planning, in-hand manipulation, visual perception, sustained attention, sensory processing, and eye-hand coordination. As a result, handwriting and handwriting analysis is often employed as a clinical test, marker, and/or indicator of an underlying cognitive or motor deficit. Several clinically significant outcomes have been described based on the analysis of participant letter, word, and sentence structure, including: letter formation (sensory awareness, kinesthesia, in-hand manipulation, motor planning, eye-hand coordination, attention, visual memory, figure ground skills); sizing (motor control, visual discrimination); line alignment (kinesthesia, spatial awareness, visual perception); capitalization (memory, form constancy); lower case letter positioning (visual spatial skills, kinesthesia, memory); letter and word spacing (visual spatial skills, left/right awareness, visual perception); letter reversal (visual perception, visual recognition, visual discrimination); missing letters (attention, visual perception); and angulation (bilateral coordination, visual perception). Given the myriad of cognitive and motor tasks required for proficient handwriting, several groups have investigated the utility of handwriting in several settings, including but not limited to:
Medical: Diagnosis, prognostication, and treatment effects in mental illness, dementia, stroke, debility, psychological disorders, emotion and personality trait deficits, and physical impairments.
Legal: Authentication of handwriting sample origins, such as needed in forensic investigations and truth telling.
Educational: Assessment of language skill, mental processing, and school/education based deficits.
Occupational: Assessment motor and/or cognitive skill or expertise.

There are education assessments which also use a series of shape sketches (a triangle made of circles, interlocking squares) to define motor and cognitive abilities. These assessments often include evaluation of both letters and sketches or non-letter image handwriting. One example of such an assessment is called the Beeri-VMI.

Significantly, the assessment of handwriting may be subjective or objective based on the analysis method used, with each method possessing its own pros and cons. While subjective analysis does not require complex tools and/or analytical methods, is inexpensive, and may meet necessary requirements for a cursory qualitative or “big picture” conclusion, it is unable to be standardized and cannot be accurately compared to prior iterations from the same participant or others for statistical analysis purposes.

Existing techniques have further established implementation of computer analysis of handwriting through the use of a digital interface to record user strokes (finger or stylus) and compare the data collected to a population of known outcomes to predict clinically relevant information. Some approaches further contribute by improving character recognition accuracy by incorporating personal writing styles and attributes into the analysis. Existing techniques have further added to handwriting analysis by incorporating coordinate systems to delineate user input positioning within the user interface. Existing techniques have further established a machine learning system by which various digital (including handwriting) and survey based responses are individually and/or collectively analyzed to predict language proficiency. Despite the successes of computerized handwriting analysis to date, current system failures include language dependence, poor translation of paper-pen outcomes with digitized handwriting samples, individual letter and word analysis with segmentation and vectoring as opposed to assessment of the product as whole, and lack of standardization as it relates to diagnosis.

### SUMMARY

Handwriting analysis as performed by analysis and machine learning. A handwriting sample is received and input. The sample is analyzed by one or more analysis components comprising criteria consisting of the group consisting of:
segmentation analysis of handwriting with numeric extraction of data,
vector analysis of handwriting,
demographic data,
known diagnoses,
data from other manual/motor tasks, and
data from other cognitive/higher function tasks; and

Machine learning is used to adjust or add criteria in at least one of the analysis components, the machine learning comprising a predicted probability of diagnosis based on prior handwriting analysis samples.

### BRIEF DESCRIPTION OF THE DRAWINGS

FIGS. 1A and 1B are schematic block diagrams, showing the technique for evaluation of handwriting and revising the evaluation of handwriting based on ongoing input of data.

FIG. 2 is a schematic block diagram, showing the process by which criteria are added.

### DETAILED DESCRIPTION

Overview

Significantly, the assessment of handwriting is augmented by an objective analysis of handwriting. Such objective analysis of handwriting offers several advantages to a subjective review. These include the ability to be standardized, the ability to be compared statistically to other samples, which are used as comparative norms, and the ability for reproducible and highly accurate results. Limitations to the objective analysis of handwriting include: mastery of complex analysis methods and algorithms, cost for analysis tools, difficulty in assessing characters across different languages (i.e., English letters vs. Hebrew characters vs. Arabic characters), slow analysis rate if not computerized or automated, and complexities required for computer/graphical analysis.

With the advent of modern computing, technology has developed to a point where systems can accurately and quickly interact with users, even adapting to the manner in which they interact with digital interfaces. With regards to the digital representation of handwriting and its incorporation into touch-screen and stylus user interfaces, computer analysis has allowed for character recognition.

Machine learning, the ability for computers to process data and learn on their own, has greatly impacted the manner and accuracy with which handwriting and digitally formatted handwriting (touch screen and stylus directed) can be analyzed and implemented for use. The disclosed techniques apply to handwritten text, hand-sketched shapes, and or a combination of the two. Specifically, the disclosed technology relates to a machine learning powered diagnostic system and method, primarily through image analysis, for predicting clinical conditions of health and illness as defined by the World Health Organization, using handwritten text and/or hand-sketched shapes as input. The International Classification of Functioning Disability and Health (ICF) as defined by the World Health Organization (WHO) includes how a person performs activities and tasks required from him in every day life and how that person behaves and participates in various environments such as home and family, work, academic and social environments and leisure activities as part of the definition of health and illness. This is in addition to the more standard definition of health and illness which previously was exclusive to body structure and function.

The disclosed techniques build upon the clinical utility of handwriting in predicting diagnoses and treatment responses through an integrated machine learning approach. While prior art has demonstrated the ability of machine learning to recognize handwritten and digitized characters, better identify user handwriting styles, and supplement language processing analysis through digitized character recognition, implementation of image analysis, as opposed to text-only extraction and analysis, for assessing handwriting has not been described. Further, while the use of handwriting for the purpose of diagnosing has been described, implementation of machine learning for this task has not. Importantly, as discussed below, machine learning directed handwriting analysis outcomes, or any machine learning directed analysis outcome, is a computer based prediction. While this prediction may be based on human directed statistical analysis, the method by which a human would calculate the final statistical prediction is inherently different than the computer. Also, as discussed, this method of analysis opens further analysis opportunities with unsupervised learning, whereby a machine learning algorithm can identify the differences in handwriting samples (pathology vs normal) without the need for human mediated constraints, such as in supervised learning methods.

The disclosed techniques include several aspects which include, but are not limited to, the following examples: Analyzing handwriting with machine learning image analysis, as opposed to segmentation, vector, or text specific analysis; incorporation of segmentation, vector, and/or text specific analysis into a machine learning image analysis system; incorporation of pre-determined classification metrics, such as objective metrics and subjective metrics, which can be obtained from direct inspection of handwriting, caliber measurements of handwriting, segmentation and vector analysis of characters, letters, and words, and/or classification of a group of handwriting samples by known or unknown pathology/disease states; previously described aspects for treatment response analysis in addition to the aforementioned method(s) for diagnosis.

Handwriting is a complex human activity, which as discussed, is often used as a surrogate test for analyzing a person's cognitive, motor, and higher-function capabilities. For example (limited), handwriting analysis has been used in the school setting to assess education and learning deficiencies in children, in the medical field as supplemental data in the diagnosis of Alzheimer's dementia or stroke recovery, and the forensic field in identifying the origins of a sample as the outward manifestation of an individual's perceptual-motor abilities and style are unique to each person. As such, different methods, with varying success, have been employed to better define accurate ways in which a diagnosis of a clinical condition can be made or enhanced via handwriting analysis. To date, population statistical analysis with handwriting sample comparison to normal or pathologic handwriting sample libraries and/or datasets has proved to be the most popular and accurate method. It is desired to further improve the utility of handwriting analysis in the diagnosis and treatment of health and wellness conditions by applying machine learning algorithms to the process.

By incorporating the pattern recognition, mathematical calculation, and graphical interpretation epitomized by computers into the handwriting analysis scheme, in combination (or not) with additional clinical and demographic data, substantial utility is demonstrated in machine learning to analyze handwritten samples for predicting those as risk for educational delays based on the underperformance of the student hand-writer when compared to their peers. This was demonstrated with both image analysis methods/modeling as well as with extracted numeric data, as detailed below in the Proof of Concept portion of this document. The disclosed technology pertains to the novel use of image analysis machine learning with or without 1) The additional use of segmentation analysis, vector analysis, and extracted numeric data analysis employed with or without machine learning and 2) The additional use of clinical and/or demographic data. The models and methods of machine learning employed for the disclosed handwriting analysis process have been described, with some models/methods commercially available (as offered by Google and IBM).

Machine learning image analysis of handwriting for the purposes of diagnosis and treatment offers significant advantages over the prior art, primarily since disease states effect the whole of the handwriting sample, not simply individual letters. Prior art established that measurable letter and word characteristics (detailed above), such as line alignment, letter and word spacing, and angulation can be collected from people with normal and disease states and statistically compared to determine what aberrations, if any, exist in these characteristics between the two that could be used to define an unknown handwritten samples as normal or pathologic. While this method offers the opportunity to test handwritten samples it is limited in the scope of what data can be extracted an analyzed. This limited scope includes, but is not limited to: 1) The metrics the practitioner is aware of—other/new metrics may exist we do not yet know, 2) The disease states being tested—handwriting may be effected by diseases not currently being studying, and 3) The method by which handwritten samples are obtained—prior art details the use of digital handwriting obtained with a touch screen or stylus may not be available in socioeconomically disadvantaged settings, may not be accessible at the time of testing, may not be optimally interacted with by users unfamiliar with modern technology, i.e., elderly people, and for which aberrations in handwriting may be secondary to the device-human interaction and not pathology. Furthermore, the limited tactile feedback associated with touch-screen and stylus interactions may limit the quality of handwriting thus impairing proper analysis or may artificially enhance handwriting via auto-smoothing algorithms thus artificially enhancing handwriting. The use of pen-paper methods for handwriting sample acquisition can help to alleviate the limitations of digital handwriting analysis acquisition, as above, though limits the utility of vector/segmentation analysis. In other prior art, pen-paper handwritten samples have been scanned and digitized for segmentation analysis and numeric data extraction. This was for the forensic analysis of handwriting to determine if the data extracted compared to others in a database for identification of the original writer. Again, this method of analysis is limited to segmentation/vector measurements which limits what can be measured and does not take into account the “style,” “spirit,” and/or overall “big-picture” of what was written—thus limiting the potential data that could be collected and analysis that could be performed.

Through the use of machine learning image analysis, where a handwriting sample is evaluated in whole or in part as a picture (image), a handwriting sample image may be compared to others, offering further insight into diagnoses or treatment responses. This method augments the data potentially available for analysis and may be combined with other, already established methods. Through the use of image classifiers, one or more identifiers can be tagged to an image and used for comparison purposes in a machine learning model. These classifiers may include normal or disease states (i.e., normal, depression, dementia), time period after intervention (i.e., immediate post stroke, 1 week after stroke therapy, 1 month after stroke therapy), functional ability (i.e., 4th grader with normal handwriting, 4th grader with abnormal handwriting), and/or a combination of classifiers including those already mentioned and/or inclusive of additional demographic classifiers, such as age, gender, current medications taken, origin of birth, and socioeconomic status. By developing a library of handwriting images taken from different disease states and classified/tagged accordingly (as detailed above) a machine learning model, or series of models can be developed where algorithms are tasked with analyzing and determining differences between classifications. Thus, when an unknown sample of interest is presented to the machine learning model(s)/system a prediction as to which outcome it best exemplifies can be made, such as 95% dementia or 86% normal. Clinicians and/or end users may then use the information as needed.

The model/system described may be combined with other methods to enhance overall performance, such as: first assess overall legibility with a text analysis module followed by image analysis for overall flow and sizing/spacing, or image analysis output in the context of gender may signify different diagnoses. As a non-limiting example, 95% in a man may indicate pathology where as 85% would be needed to define pathology in a woman. The methods detailed here fall under the category of supervised machine learning, where model constraints (i.e., classifiers) are used to guide the learning process. In other methods of machine learning, namely unsupervised learning, no initial input is made to the models and the machine learning algorithm(s) are capable of determining which samples are different from others. Following, clinical input can be made to predict why. As a non-limiting example, a determination may comprise what it means that handwriting samples labeled A are different from B and C.

There are various approaches to implementing the disclosed techniques. In one configuration for implementing the disclosed techniques the diagnosis of health and illness comprises an illness, a mental illness, a physiological condition, a mental or emotional state, medication effect, a personality trait, a skill, an expertise, deception or truth writing, or any combination thereof. In one configuration for implementing the disclosed techniques the handwriting image classification for a supervised model is based on an initial analysis of handwriting character, letters, words, sentences, and sketches relating to analysis of pen stroke, spatial relationships, letter formation, alignment, spacing, angulation, length, trajectory, silhouette of handwritten shape, height, width, and alignment. In one configuration for implementing the disclosed techniques the handwritten image classification for a supervised model is based on pre-recorded diagnoses or recorded indicators for the sample.

In one configuration for implementing the disclosed techniques the handwritten image classification is established by giving a set of handwriting tasks to a first group known to have a certain condition and giving the same set of handwriting tasks to a control group known not to have a condition (normal) and analyzing the recorded indicators and/or extracting numerical values of the handwriting samples to determine threshold values that differentiate the two. This is followed by supervised learning of a machine learning model where the classifiers are used to define image populations for predicting the diagnosis from handwriting samples of unknowns.

The disclosed techniques can incorporate classifying subjects according to age and education level norms for the purposes of supervised machine learning where outcomes of good/bad or pass/fail are defined by the dominant predicted grade/age/level from the machine learning algorithm as compared to the input sample. As a non-limiting example, a 4th grade student handwriting sample in a school based screening program is predicted to be at a 2nd grade level by the machine learning algorithm and thus the student's handwriting screening outcome is “fail”.

The disclosed techniques' handwriting tasks can involve writing letters, numbers, drawings or any combination thereof on a pen-paper sample. The disclosed techniques' handwriting tasks can involve writing letters, numbers, drawings or any combination thereof on a touch screen or stylus system. The disclosed techniques' handwritten tasks are classified via validation and comparison to known standardized samples. The disclosed techniques' handwritten task can be functional and associated with everyday requirements such as name writing, copying text, and writing from memory. Similarly, the disclosed techniques' handwriting tasks may include drawing/sketching/copying complex figures or drawing of figures such as a clock, person, tree, or home. Comparison of handwriting results between normal and disease states may then be used to define classifiers. Comparison of handwriting results between unknown samples may be used to identify subpopulations with disease states that must be determined later one. As a non-limiting example, the unknown samples may be unknown at the time of acquisition (unsupervised learning).

The analysis can be performed by discrete analysis and by machine learning. In doing so, a sample input is input to a computer, either directly or by scanning the sample as an image. The machine analysis is performed according to defined analysis criteria, and also by machine analysis of the handwriting sample as a whole. In this way, the handwriting sample can be analyzed as extracted data as well as analysis of the handwriting image as a singular unit. Thus a machine learning analysis can be performed on one or a combination of pre-defined items, one of which is of the image as a whole.

In one configuration for implementing the disclosed techniques' handwriting classification and machine learning analysis uses additional tags to classify or stratify images based on personal information, such as demographic data, known disease states, known medication therapies, and/or other questionnaire data. The disclosed techniques' machine learning analysis of handwriting samples will be used in conjunction with other established methods such as statistical analysis of vector or segmented portions of handwriting, text analysis (machine learning or otherwise), and multiple machine learning algorithms. The disclosed techniques' machine learning algorithms used are commercially available. Alternatively, the disclosed techniques' machine learning algorithms may be generated independently by someone with knowledge of the art. The techniques for data collection, feature extraction, and statistical analysis by hand or computer segmentation/vector may be employed to define handwriting characteristics as normal or not for use in classification models.

The disclosed techniques' handwriting samples may be composed of text such as, by way of a non-limiting example, a same single sentence or may use a sketch/drawing across all samples on a pen-paper model which is scanned into a computer as a pdf or image document. The image may be analyzed by feature extraction to define the sample for classification models or features may be used to further define handwriting samples before or after machine learning analysis to enhance output predictions of disease/pathology. The disclosed techniques' machine learning portion of the analysis may be employed using a graphic user interface, command lines, or the like to upload images and receive the results. Further, the interface may use the following, though not limited to: text boxes, graphical regions, dialogue boxes, static controls, drop down menus, list boxes, radio buttons, edit controls, push button, check boxes, graphic boxes.

The disclosed techniques' recognizer carries the burden of distinguishing good/normal groupings from bad/pathologic groupings and also assigns correct labels to good and bad groupings. These labels can then be used as classification labels for image based machine learning for making diagnostic predictions based on the handwriting samples.

The disclosed techniques can use a skill model that can comprise a statistical model identifying a predictive skill level of one or several users. As a non-limiting example, the disclosed techniques may be used in an educational setting by predicting handwriting performance by grade. The disclosed techniques' diagnosis system may comprise the above with the addition of demographic and or clinical data for model optimization.

The end-user can upload a handwriting image to a cloud/server based machine learning model, encrypted or unencrypted protection employed, and receive the output of handwriting samples via email, text, fax, downloadable file/table, or presented on the computer screen.

In one configuration for implementing the disclosed techniques, the diagnosis system may comprise: data collection, data analysis of certain handwriting characteristic(s), use of these characteristics to train a machine learning model, optimization of the model, and having the model predict disease from normal states. The disclosed techniques' diagnosis system may comprise: data collection and unsupervised machine learning to predict differences between groups of data to be determined by the end user as to the implications.

The disclosed handwriting analysis technique has several components:
1. Obtain or retrieve a writing sample.
2. Identify particular aspects of the writing sample. The particular aspects can be previously-identified characteristics that may in at least some instances indicate a neuromuscular condition. Alternatively, the particular aspects can be generalized characteristics evident in some people's handwriting.
3. Create associations between particular aspects and other particular aspects found in the subject's handwriting.
4. Identify the particular aspects with at least one neuromuscular condition, condition of other pathology, or general aspects of writing style by the subject.
5. Use machine learning to make predictions regarding an outcome about the aspects and their associations to previously observed/leaned aspects. This can be used to generate/produce an outcome, prediction, and/or diagnosis.
6. Use machine learning to make predictions regarding an outcome about the aspects and their associations to previously observed/leaned aspects to correlate the aspects and associations with previously-observed neuromuscular conditions.

By use of machine learning, features of different subjects' handwriting that correlate to a neuromuscular response can be analyzed. The neuromuscular response can be correlated with pathological conditions, and in some cases may be used in the diagnosis of the pathology. In some cases, the condition being diagnosed may not be apparent to a careful observer, but may nevertheless be present in the handwriting. Moreover, different conditions are affected by different external factors, e.g., alcohol and other drugs. If the condition can be identified at a stage in which the condition is not apparent, but can be confirmed, then that data can be “learned” as generally consistent with the identified pathology. The detection of neuromuscular response can further be used to compare “response” as pathology (tremor) and treatment effect (e.g., tremor got better on new medication).

Taking this further, a machine-learned correlation can be applied at a lower level, in which observation is unlikely to confirm the condition, but a prediction can be made based on the reliability of more significant correlations. On the other hand, if a lower level correlation expands to a large percentage of the population, the prediction would not be made, or alternatively, it would be presumed that the lower level represents the typical neurology or cognitive pathology of the population or is otherwise not significant.

In addition, the technique can be used as a training tool for subjects to improve handwriting. To the extent that particular characteristics of the subject's handwriting can be identified, the subject can be given information as to what motor skills are involved and be provided with suggestions on how to improve the motor skills.

Due to the complexity of handwriting, it is possible to compare handwriting samples of the same people to determine subtle effects of various stimulants (drugs, alcohol, lighting) on the subjects. This would establish a high level of correlation which could not be achieved by standard observation.

Data Assessment and Machine Learning

The handwriting assessment provides a diagnosis system which uses handwriting analysis. The handwriting analysis comprises one more components of which at least one component uses machine learning analysis of handwriting. The other components may include:
segmentation analysis of handwriting with numeric extraction of data,
vector analysis of handwriting,
demographic data,
known diagnoses,
data from other manual/motor tasks, and
data from other cognitive/higher function tasks.

By way of non-limiting example, the diagnosis criteria itself may be defined by the International Classification of Functioning Disability and Health (ICF) as defined by the World Health Organization (WHO). This diagnosis criteria includes, but is not limited to, how a person performs activities and tasks required from him in everyday life and how that person behaves and participates in various environments such as home and family, work, academic and social environments and leisure activities as part of the definition of health and illness. This is in addition to the more standard definition of health and illness which is generally focused on body structure and function.

Examples of the diagnostic criteria include, but is not limited to, education learning deficits, education performance deficits, inability to perform activities of daily living, depression, mental illness, dementia, motor diseases such as Parkinson's, occupation performance target, and drug therapy response.

The definition of handwriting encompasses any writing or drawing activities using any of a large number of media. This may include, by way of non-limited example, pen to paper or electronic acquisition (i.e., a touch screen tablet) for writing letters, writing numbers, drawing simple figures, drawing complex figures, and/or any combination of the aforementioned. As used herein, “pen” is intended to describe a writing instrument intended for manual writing, such as a pen, but can also reference a pencil or other writing instrument, as well as a computer simulation of a manual writing instrument. The handwriting analysis systems seeks to determine or predict the probability of a diagnosis using a handwriting sample obtained via the described methods.

The determination and or prediction of diagnosis may be based on several methods, including but not limited to:
degrees or separation or variance from normal using extracted data and statistical analysis,
comparison of handwriting samples of normal and abnormal “knowns” with unknown samples to generate a prediction,
direct measurement of handwriting using calipers, and
conversion of the handwriting to an image for analysis.

The disclosed technique uses a computer analysis, which is enhanced by machine learning. Specifically machine learning analysis is defined by any method, commercial or otherwise, which uses machine learning algorithms and/or artificial intelligence (AI) to generate some if not all of the predictions of diagnosis based on all or part of the handwriting sample. Supervised training data sets may be obtained by sorting handwriting samples based on known information about patients; i.e., normal and abnormal. For diagnosis purposes, “abnormal” can be defined by already predetermined diagnosis, and can refer only to the specific function or response being tested, as opposed to categorizing the subject as “abnormal”. “Abnormal” may also be defined by degrees of variance between images of handwriting. “Abnormal” may also be defined by expert clinical evaluation of samples.

Handwriting samples are then classified as normal or disease state using any method(s) chosen by the user for supervised training of models.

Examples of evaluation may comprise, by way of non-limiting example, evaluation by an occupational therapist or other handwriting expert. Unsupervised training of machine learning models may also be used to define handwriting samples that differ from others. This is followed by analysis of the associated patients to determine a diagnosis.

Predetermined test criteria or components of predetermined test criteria may be used before, after, or in conjunction with the machine learning analysis. The components may be connected to one another in several ways, examples including but not limited to: A cohesive computer program, electronic health record, electronic server, decision tree algorithm, step-wise algorithm.

The data collected from the patients, including demographic, diagnosis, and handwriting extracted data may be presented and/or stored in a data table. The data collected and handwriting images may be collected and stored as a single unit per patient and organized by a single or multimode machine learning algorithm.

The machine learning analysis constitutes image analysis of the handwriting samples. The handwriting sample acquired by pen to paper is converted to an image file. As indicated, “pen” is intended to indicate the use of any writing or drawing activities using any of a large number of media. This may include, by way of non-limited example, pen to paper or electronic acquisition (i.e., a touch screen tablet) for writing letters, writing numbers, drawing simple figures, drawing complex figures, and/or any combination of the aforementioned. Alternatively, the handwriting analysis may be acquired by electronic interaction, such as through a touch screen, is collected as a whole and converted to an image.

The output provides a predicted diagnosis from a handwriting analysis system, which can be used to guide therapy. By way of non-limiting example, the approach to therapy can be general analysis or can provide specific information such as which medications to use, positive or negative response to medications, which interventions/physical/occupational therapy to use, and positive or negative response to therapy.

The output provides a predicted risk for future diagnosis, such for forecasting an impending or distant outcome, from a handwriting analysis system which can be used to risk stratify patients and/or guide therapy and/or intervention. By way of non-limiting example, a handwriting screen may be developed where the risk for a future outcome, such as Alzheimer's disease, may be made. In this context, a patient may be told they have a high risk of developing the disease which may warrant more frequent screening and/or preemptive treatment strategies.

Process

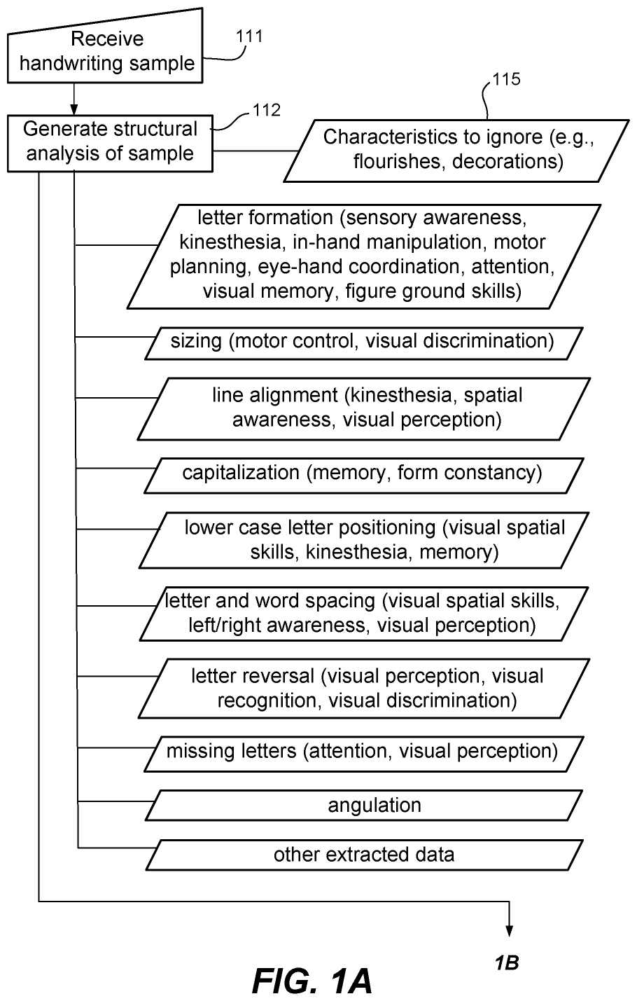

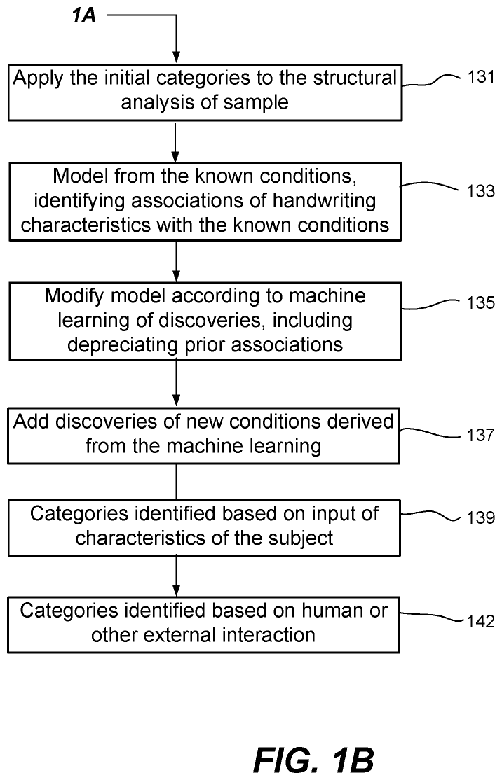

FIGS. 1A and 1B are schematic block diagrams, showing the technique for evaluation of handwriting and revising the evaluation of handwriting based on ongoing input of data. The handwriting sample is received (step 111), and structural analysis of the sample is generated (step 112). Using the structural analysis, the sample is evaluated according to initial categories, representing known conditions. Characteristics to ignore are excluded from the evaluation (step 115).

Non-limiting examples of initial categories can include such characteristics as:

| | |
|---|---|
| letter formation | possibly associated with sensory awareness, |
| | kinesthesia, in-hand manipulation, motor |
| | planning, eye-hand coordination, attention, |
| | visual memory, figure ground skills |
| sizing | possibly associated with motor control, |
| | visual discrimination |
| line alignment | possibly associated with kinesthesia, spatial |
| | awareness, visual perception |
| capitalization | possibly associated with memory, form |
| | constancy |
| lower case letter | possibly associated with visual spatial skills, |
| positioning | kinesthesia, memory |
| letter and word spacing | possibly associated with visual spatial skills, |
| | left/right awareness, visual perception |
| letter reversal | possibly associated with visual perception, |
| | visual recognition, visual discrimination |
| missing letters | possibly associated with attention, visual |
| | perception |
| angulation | |

In addition to the initial categories, it is expected that other categories will be developed, either through experience or through machine learning. The depiction includes letter and word specific characteristics, which is used as a non-limiting example for current handwriting analysis. There are many other attributes which could be used to analyze handwriting, some of which may not be immediately apparent, but which can be useful from an “extracted data” standpoint.

A handwriting analysis model is created (step 131) from the known conditions, identifying associations of handwriting characteristics with the known condition (step 133). The associations can be identified based on input of characteristics of the subject, or can be identified based on human or other external interaction. The analysis is modified (step 135) according to machine learning of discoveries, or of depreciating prior associations. Since additional data is received which can include handwriting characteristics (step 137) and the subject's conditions, the additional data can be used to establish its own categories (step 139). The new categories may be self-identified, as a result of the condition having been identified, or as a result of the handwriting characteristic itself having been detected.

In response the handwriting analysis model is modified or expanded, and if necessary, a manual attempt is made to identify conditions associated with the handwriting characteristic (step 142). Additionally, since additional information is received, it is possible that prior associations between the handwriting characteristics and the subjects' conditions may be depreciated.

The identified associations of handwriting characteristics and the establishment of new categories allows new conditions to be derived from the machine learning. In some cases, the new conditions will be non-consequential; however it is possible that conditions that are generally undiagnosed at early stages can be identified.

In analyzing and processing the associations, some aspects of handwriting are ignored as being non-consequential or irrelevant to handwriting analysis. Examples are flourishes and decorations, which, while possibly relevant to identification, do not provide other useful associations.

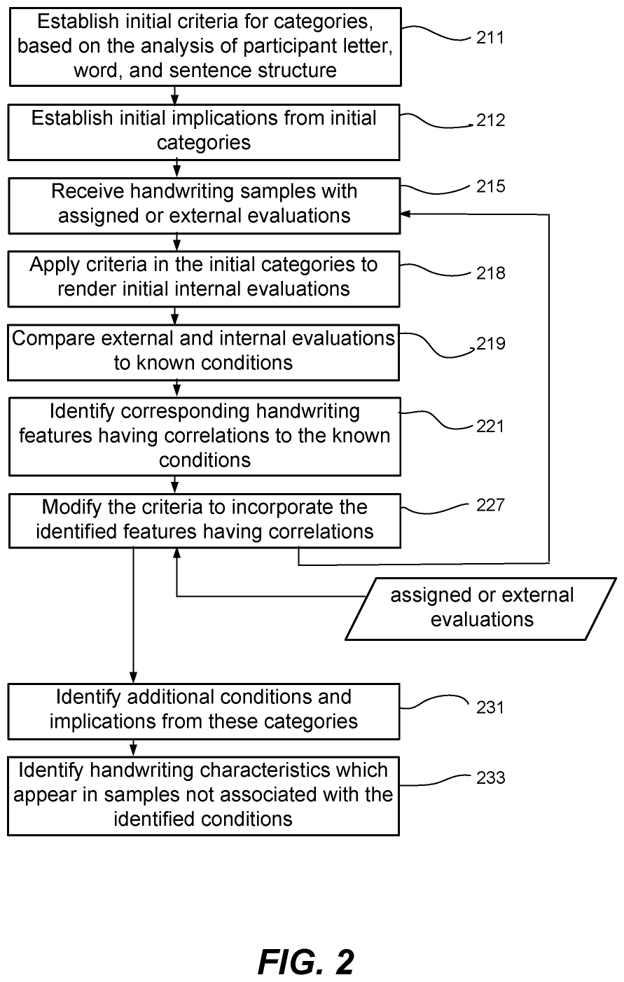

FIG. 2 is a schematic block diagram, showing the process by which criteria are added. The initial criteria for categories are established (step 211), based on the analysis of participant letter, word, and sentence structure. Initial implications are established (step 212) from initial categories. Handwriting samples are received (step 215) and the samples are subjected to a structural analysis. Initial implications from initial categories are established (step 218). Handwriting samples with assigned or external evaluations may also be received (step 219).

Using the handwriting samples, criteria in the initial categories are applied (step 221) to render initial internal evaluations. External and internal evaluations to known conditions are compared, and corresponding handwriting features having correlations to the known conditions are identified. The criteria are modified (step 227) to incorporate the identified features having correlations. Here we are describing letters and words. It is also possible for someone to handwrite something else, for example a cluster of circles or a series of lines, or a sketch which could also be analyzed by machine learning for predicting an outcome.

This is repeated in a loop beginning with receiving handwriting samples (step 215) with assigned or external evaluations. Additional conditions and implications from these categories are identified (step 231). Handwriting characteristics which appear in samples not associated with the identified conditions are then identified (step 233).

Implementation and Proof of Concept

A total of 1,906 handwriting samples from grades K-6 from schools in urban and sub-urban locations within New York State were obtained following external IRB protocol review. The handwriting samples were acquired on identical intake forms and had students copy the same sentence onto wide space ruled lines on the form. The handwriting acquisition was performed by students' teachers in a manner most similar to the way any other school based assignment would have been provided, per the teacher's standard classroom protocol. A licensed pediatric occupational therapist graded all handwriting samples. Initially, 100 samples were selected at random and objective measurements of 11 letter and word characteristics were made, including letter size and word spacing. The mean and standard deviation values for these metrics were calculated. For assessment of the remaining samples, errors in handwriting were defined by measurements of these 11 attributes for all letters and words provided that were greater than two standard deviations above or below the mean. The 11 objective measurements were selected from literature sources detailing the relationship between these metrics and the following handwriting associated cognitive and motor activities: Fine motor control, gross motor control, spatial awareness, pre-planning, eye-hand coordination, and cognition. A subjective measurement of letter legibility was made by the occupational therapist.

Following, a stochastic modeling researcher received a data set with total errors per handwriting sample as defined by the occupational therapist. This data analyst was blinded to the actual handwriting samples. A series of student T-tests was performed comparing to the total number of errors per handwriting samples between grades. These initial tests demonstrated that kindergarten, first grade, second grade, and third through sixth grades (as a combined group) were statistically different from one another. Accordingly, sub-par handwriting threshold values for errors/sample were defined for each of these four groups. Mean, standard deviation, and histogram analysis was used to define student populations within each group which were at least one standard deviation below the mean for errors/sample. Thus, the following percentiles were used to define sub-par handwriting:

| | |
|---|---|
| kindergarten | 27th percentile |
| first grade | 20th percentile |
| second grade | 14th percentile |
| third through sixth grade | 11th percentile |

Based on the threshold values determined above, each handwriting sample was assigned an outcome for use in the machine learning tests—good and bad. Following scanning to PDF, conversion to an image file, cropping images so only the handwritten sample was present, and separating handwriting samples into pass/fail categories based on the error percentiles above, machine learning algorithm platforms were tested for their ability to learn and differentiate data between the good and bad samples. Model performance demonstrated above 65% precision within all models tested. In other models, where only the extracted numeric data was obtained and data rows (individual handwriting samples) labeled as pass/fail based on the grade based error percentiles above, multilayer perceptron modeling reached near 100% sensitivity and specificity for predictions based on extracted data alone. When models were applied to whole images of handwriting samples, sensitivity and specificity ranged between 62-85% and 71-96%, respectively, based on grades. Note, that currently accepted screening tests for RTI schools (Response to Intervention) have sensitivity and specificity metrics which range between 26-92% and 63-96%, respectively.

### CLOSING STATEMENT

It will be understood that many additional changes in the details, materials, steps and arrangement of parts, which have been herein described and illustrated to explain the nature of the subject matter, may be made by those skilled in the art within the principle and scope of the invention as expressed in the appended claims.

What is claimed is:

1. A method for providing handwriting analysis comprising:
receiving and inputting a handwriting sample;
analyzing the handwriting sample by one or more analysis components comprising criteria consisting of the group consisting of:
segmentation analysis of handwriting with numeric extraction of data,
vector analysis of handwriting,
demographic data,
known diagnoses,
data from other manual/motor tasks, and
data from other cognitive/higher function tasks; and
using machine learning to adjust or add criteria in at least one of the analysis components, the machine learning comprising a predicted probability of diagnosis based on prior handwriting analysis samples.

2. The method as described in claim 1, further comprising:
the machine learning analysis comprises machine learning algorithms and/or artificial intelligence to generate some if not all of the predictions of diagnosis based on all or part of the handwriting sample, said predictions of diagnosis comprising:
supervised training data sets may be obtained by sorting handwriting samples based on known information about patients,
abnormal results defined by already predetermined diagnosis,
abnormal results defined by degrees of variance between images of handwriting,
abnormal results defined by expert clinical evaluation of samples (such as by an occupational therapist or other handwriting expert), and
unsupervised training of machine learning models may also be used to define handwriting samples that differ from others; and
subsequent to using the machine learning analysis, providing analysis of the associated patients to determine a diagnosis.

3. The method as described in claim 1, further comprising:
wherein the handwriting analysis comprising a diagnosis is defined by the International Classification of Functioning Disability and Health (ICF) as defined by the World Health Organization (WHO).

4. The method as described in claim 1, further comprising:
wherein the handwriting analysis comprises education learning deficits, education performance deficits, ability or inability to perform activities of daily living, depression, mental illness, dementia, motor diseases, occupation performance, and drug therapy response.

5. The method as described in claim 1, wherein handwriting comprises writing or drawing activities, the writing or drawing activities comprising pen or other manual response handwriting tool to paper, electronic acquisition, electronic acquisition for writing letters, writing numbers, drawing simple figures, and drawing complex figures.

6. The method as described in claim 1, wherein the handwriting analysis systems seeks to determine or predict the probability of a diagnosis according to said one or more analysis components.

7. The method as described in claim 1, further comprising using the handwriting analysis systems to determine or predict the probability of a diagnosis according to said one or more analysis components, not limited to: degrees or separation or variance from a predetermined normal obtained by using extracted data and statistical analysis, comparison of handwriting samples of normal and abnormal known criteria with unknown samples to generate a prediction.

8. The method as described in claim 1, wherein the machine learning comprises using the segmentation analysis to collect data from the samples to then classify the samples to train a machine learning model so as to make predictions on other samples.

9. The method as described in claim 1, wherein the machine learning comprises using the segmentation analysis as part of the analysis in conjunction with machine learning to make a diagnosis.

10. A method for providing handwriting analysis comprising:
obtaining a writing sample;
receiving and inputting the writing sample into a non-transient computer-readable medium;
and analyzing the input writing sample to obtain a diagnosis; and
using machine learning to predict a probability of diagnosis.

11. The method of claim 10, further comprising:
identifying particular aspects of the writing sample;
establishing associations of the particular aspects with other particular aspects found in the subject's handwriting;
identifying the particular aspects with at least one of neuromuscular conditions, cognitive disorders, sensory disorders and general aspects of writing style by the subject;
using the machine learning to correlate the aspects and associations with previously-observed neuromuscular conditions.

12. The method of claim 9, further comprising using the particular aspects of the writing sample to identify characteristics that may in at least some instances indicate a neuromuscular condition.

13. The method of claim 9, further comprising using the particular aspects of the writing sample to identify characteristics that may in at least some instances indicate a neuromuscular condition or a generalized characteristic evident in a subject's handwriting.

14. A method for providing handwriting analysis comprising:
receiving and inputting a handwriting sample;
generating an initial structural analysis of the handwriting sample, the initial structural analysis comprising plural predetermined components of the handwriting sample;
applying the initial categories to the structural analysis of sample;
modelling a handwriting analysis from known conditions associated with the predetermined components of the handwriting sample;
modifying the model according to machine learning of discoveries, said modifying comprising depreciating prior associations according the machine learning;
adding discoveries of new conditions derived from the machine learning;
identifying categories based on an input of characteristics of a subject providing the handwriting sample;
identifying categories based on human or other external interaction.

15. The method of claim 14, wherein the plural predetermined components of the handwriting sample comprise one or more of:
letter formation,
sizing,
line alignment,
capitalization,
lower case letter positioning,
letter and word spacing,
letter reversal,
missing letters, and
angulation.

16. The method of claim 14, further comprising:
establishing initial criteria for the categories based on an analysis of letter, word and sentence structure of the received handwriting sample;
establishing initial implications from the initial categories;
receiving handwriting samples with assigned or external evaluations;
comparing external and internal evaluations to known conditions;
identifying corresponding handwriting features having correlations to the known conditions;
modifying the criteria to incorporate the identified features having the correlations;
identifying additional conditions and implications from the categories; and
identifying handwriting characteristics which appear in samples not associated with the identified conditions.

17. The method of claim 14, further comprising:
analyzing the handwriting sample by one or more analysis components comprising criteria consisting of the group consisting of:
segmentation analysis of handwriting with numeric extraction of data,
vector analysis of handwriting,
demographic data,
known diagnoses,
data from other manual/motor tasks, and
data from other cognitive/higher function tasks; and
using machine learning to adjust or add criteria in at least one of the analysis components, the machine learning comprising a predicted probability of diagnosis based on prior handwriting analysis samples.
